(19) United States
(12) Patent Application Publication
(10) Pub. No.: US 2005/0252281 A1
(43) Pub. Date: Nov. 17, 2005

(54) System and method for treating process fluids delivered to an electrochemical cell stack

(76) Inventors: Ralph Stanley Worsley, Brentwood Bay (CA); Gordon John Lamont, New Westminster (CA); Jacek P. Piotrowski, North Saanich (CA); Paul Kenneth Adam, Port Moody (CA)

Correspondence Address: BERESKIN AND PARR, 40 KING STREET WEST, BOX 401, TORONTO, ON, M5H 3Y2, CA

(21) Appl. No.: 11/013,348

(22) Filed: Dec. 17, 2004

Related U.S. Application Data
(60) Provisional application No. 60/529,759, filed on Dec. 17, 2003.

(51) Int. Cl. B01D 37/00; G01N 33/00

(52) U.S. Cl. 73/61.41; 210/600

(57) ABSTRACT

A system for treating process fluids delivered to an electrochemical cell stack is described. The system includes a treatment unit capable of treating and imparting a first range of temperatures to a process fluid that includes the freezing point of water. A filter removes ice particles larger than a particular size formed when the first temperature unit imparts a temperature to the process fluid low enough to cause ice particles to form.

### FIELD OF THE INVENTION

The present invention relates to electrochemical cell stacks, and more specifically to the treatment of process fluids delivered thereto.

### BACKGROUND OF THE INVENTION

Electrochemical cell stacks include fuel and electrolytic cell stacks. A fuel cell is an electrochemical device that produces an electromotive force by bringing a fuel (typically hydrogen gas) and an oxidant (typically air or oxygen gas) into contact with two suitable electrodes and an electrolyte. The fuel is introduced at a first electrode where it reacts electrochemically in the presence of the electrolyte to produce electrons and cations. The electrons are circulated from the first electrode to a second electrode via an electrical circuit. Cations pass through the electrolyte to the second electrode.

Simultaneously, the oxidant is introduced to the second electrode where the oxidant reacts electrochemically in presence of the electrolyte and catalyst, producing anions and consuming the electrons circulated through the electrical circuit; the cations are consumed at the second electrode. The anions formed at the second electrode or cathode react with the cations to form a reaction product. The first electrode or anode may alternatively be referred to as a fuel or oxidizing electrode, and the second electrode may alternatively be referred to as an oxidant or reducing electrode.

The half-cell reactions at the two electrodes are, respectively, as follows:
H₂→2H++2e−
½O₂+2H++2e−→H₂O
The external electrical circuit withdraws electrical current and thus receives electrical power from the fuel cell. The overall fuel cell reaction produces electrical energy as shown by the sum of the separate half-cell reactions written above. Water and heat are typical by-products of the reaction.

Conceptually, electrolytic cells, or electrolyzers, are fuel cells run in reverse, and share many of the same components as fuel stacks. In particular, a current is supplied to the electrolytic cell stack for the electrolysis of water into hydrogen and oxygen gases. In a fuel cell, hydrogen and oxygen are combined to produce water and release heat. In an electrolytic cell stack, energy is required to break up water into hydrogen and oxygen.

In practice, fuel cells are not operated as single units. Rather, fuel cells are connected in series, stacked one on top of the other, or placed side by side, to form what is usually referred to as a fuel cell stack. As used herein, the term “cell stack” includes the special case where just one fuel cell is present, although typically a plurality of fuel cells are stacked together to form a cell stack. The fuel and oxidant are directed through manifolds to the electrodes, while cooling is provided either by the reactants or by a cooling medium. Also within the stack are current collectors, cell-to-cell seals and insulation, with required piping and instrumentation provided externally of the fuel cell stack.

A fuel cell stack includes two end plates that sandwich components of the fuel cell stack. End plates provide integrity to the fuel cell stack by acting as an anchor for rods or bolts that are used to compress together various components of the cell stack resting between the end plates. Moreover, end plates can contain connection ports to which are attached fuel, oxidant and coolant ducts or hoses. These process fluids flow through the connection ports into and out of the fuel cells stack. In addition, end plates have components that insulate electrically conductive parts from parts meant to be non-conductive.

With so many components, cell stacks are periodically tested to ensure proper functioning. For this purpose, a fuel cell testing station can be used. A fuel cell test station simulates operating conditions for the fuel cell being tested and monitors various parameters indicating the performance of the fuel cell. For example, a fuel cell testing station is usually capable of supplying reactants, e.g. hydrogen and air, and/or coolant, to the fuel cell at various temperatures, pressures, flow rates and/or humidity. A fuel cell test station may also change the load of the fuel cell and hence change the voltage output and/or current of the fuel cell. A fuel cell test station monitors individual cell voltages within a fuel cell stack, current flowing through the fuel cell, current density, temperature, pressure or humidity at various points within the fuel cell. Such fuel cell test stations are commercially available from Hydrogenics Corporation in Mississauga, Ontario, Canada, or Greenlight Power Technologies in Burnaby, B.C, Canada.

Enhancing the performance of a fuel cell stack is sometimes dependent on the ability to test such performance. Thus, any innovation that can improve a cell stack test station would be most welcome in the field of electrochemical cell stack technology.

### SUMMARY OF THE INVENTION

A treatment system for treating process fluids in a fuel cell test station connected to a cell stack is described herein. Process fluids can be treated to simulate the condition of the fluids in realistic situations. Properties of process fluids that can be varied by the treatment system include temperature, pressure and humidity.

The treatment system for treating process fluids delivered to an electrochemical cell stack includes a first treatment unit capable of treating and imparting a first range of temperatures to a process fluid. This first range includes temperatures that would cause water vapour to freeze into ice particles. The treatment system also includes a filter for removing ice particles larger than a particular size formed when the first treatment unit imparts a temperature to the process fluid low enough to cause ice particles to form. At least one process fluid conduit then delivers the filtered process fluid to the cell stack.

Also described herein is a treatment system for treating process fluids delivered to a plurality of electrochemical cell stacks. Advantageously, this embodiment of the treatment system need be equipped with only one chiller device. The system also includes a plurality of heating and cooling modules, each one capable of independently setting a temperature of a process fluid therein. A filter is provided in each of the treatment systems. Each one is associated with a heating and cooling module and each one functions to remove ice particles formed when the associated heating and cooling module imparts a temperature to the process fluid low enough to cause ice particles to form. A plurality of process fluid conduits transport filtered process fluid from respective filters to the associated electrochemical cell stacks. The chiller device removes heat from the plurality of heating and cooling modules.

It is expected that this embodiment of the invention will have particular applicability to a plurality of test stations, where a common chiller device serves to providing cooling capacity to a plurality of test stations, each of which cools a process gas or fluid for a fuel cell stack or power module (i.e. a fuel cell stack and associated balance of plant components) to be tested.

The present invention also provides a method of treating a process fluid, to be delivered to an electrochemical cell stack, the method comprising: cooling the fluid to a temperature below the freezing point of water; filtering out any ice particles that have formed due to the cooling step; and delivering the process fluid to the electrochemical cell stack.

The method can be carried out in a test station. Where a plurality of test stations are provided, the method can include providing a common chilling device providing a chilled heat exchange fluid to each test station.

### BRIEF DESCRIPTION OF THE DRAWINGS

For a better understanding of the present invention, and to show more clearly how it may be carried into effect, reference will now be made, by way of example, to the accompanying drawings, which show preferred embodiments of the present invention and in which:

FIG. 1 shows an exploded perspective view of an electrochemical cell stack;

FIG. 2 shows a block diagram of a system for treating process fluids delivered to an electrochemical cell stack;

FIG. 3 shows a block diagram of another embodiment of a system for treating process fluids delivered to an electrochemical cell stack;

FIG. 4A shows a block diagram of the treatment unit of FIG. 2;

FIG. 4B shows a block diagram of another embodiment of the treatment unit of FIG. 2; and

FIG. 5 shows a block diagram of a system for treating process fluids delivered to an electrochemical cell containing one chiller device and multiple heating and cooling units.

### DETAILED DESCRIPTION OF THE INVENTION

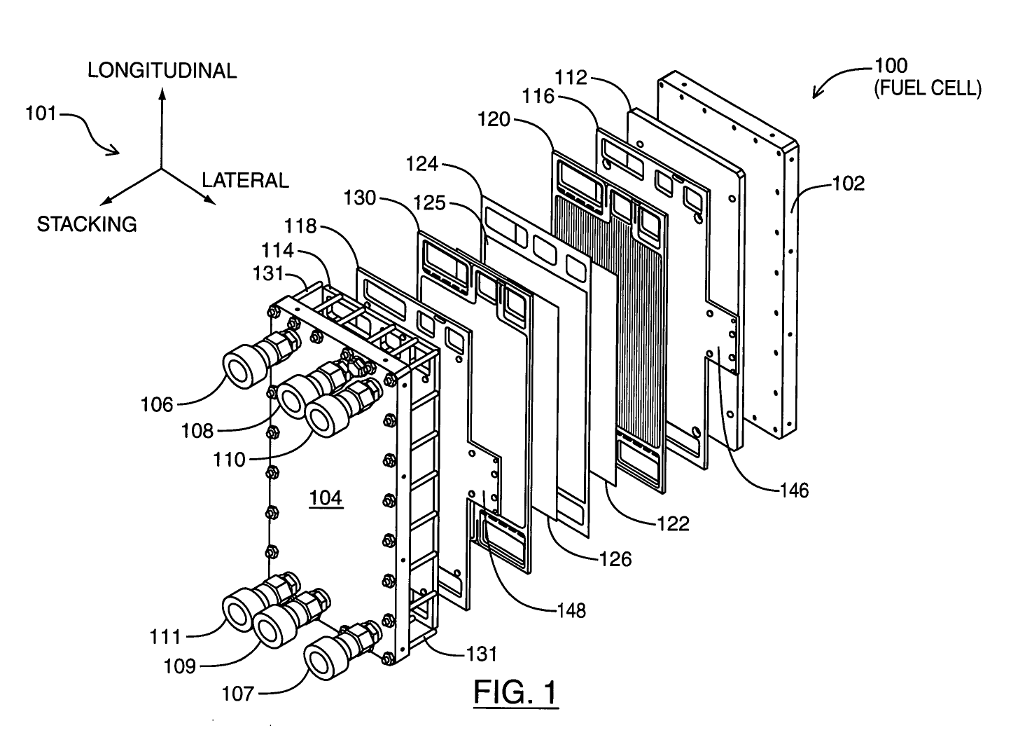

FIG. 1 shows an exploded perspective view of an electrochemical cell stack 100. A coordinate system 101, with stacking, longitudinal and lateral directions marked, is provided for convenient referencing. The fuel cell unit 100 includes an anode flow field plate 120, a cathode flow field plate 130 that sandwich a membrane electrode assembly (MEA) 124. Various sizes are possible for the plates 120 and 130. In one embodiment, for example, the short edge of the flow field plates 120, 130 is about 12 cm. Each plate 120 and 130 has an inlet region, an outlet region, and open-faced channels (not shown). The channels fluidly connect the inlet region to the outlet region, and provide a way for distributing the reactant gases to the outer surfaces of the MEA 124.

The MEA 124 comprises a solid electrolyte (i.e. a proton exchange membrane or PEM) 125 disposed between an anode catalyst layer (not shown) and a cathode catalyst layer (not shown). A first gas diffusion layer (GDL) 122 is disposed between the anode catalyst layer and the anode flow field plate 120, and a second GDL 126 is disposed between the cathode catalyst layer and the cathode flow field plate 130. The GDLs 122, 126 facilitate the diffusion of the reactant gas, either the fuel or oxidant, to the catalyst surfaces of the MEA 124. Furthermore, the GDLs enhance the electrical conductivity between each of the anode and cathode flow field plates 120, 130 and the membrane 125.

A first current collector plate 116 abuts against the rear face of the anode flow field plate 120, where the term “rear” indicates the side facing away from the MEA 124. Likewise, the term “front” refers to the side facing the MEA. A second current collector plate 118 abuts against the rear face of the cathode flow field plate 130. Each of the first and second current collector plates 116 and 118 respectively has a tab 146 and 148 protruding from the side of the fuel cell stack. First and second insulator plates 112 and 114 are located immediately adjacent the first and second current collector plates 116, 118, respectively. First and second end plates 102, 104 are located immediately adjacent the first and second insulator plates 112, 114, respectively. Pressure may be applied on the end plates 102, 104 to press the unit 100 together. Moreover, sealing means are usually provided between each pair of adjacent plates. Preferably, a plurality of tie rods 131 may also be provided. The tie rods 131 are screwed into threaded bores in the anode endplate 102, and pass through corresponding plain bores in the cathode endplate 104. Fastening means, such as nuts, bolts, washers and the like are provided for clamping together the fuel cell unit 100.

The end plate 104 is provided with a plurality of connection ports for the supply of various fluids. Specifically, the second endplate 104 has first and a second air connection ports 106, 107, first and second coolant connection ports 108, 109, and first and second hydrogen connection ports 110, 111. The MEA 124, the anode and cathode flow field plates 120, 130, the first and second current collector plates 116, 118, the first and second insulator plates 112, 114, and the first and/or second end plates 102, 104 have three inlets near one end and three outlets near the opposite end, which are in alignment to form fluid ducts for air as an oxidant, hydrogen as a fuel, and a coolant. Also, it is not essential that all the outlets be located at one end, i.e., pairs of flows could be counter current as opposed to flowing in the same direction. The inlet and outlet regions of each plate are also referred to as manifold areas. Although not shown, it will be understood that the various ports 106-111 are fluidly connected to ducts that extend along the stacking direction of the fuel cell unit 100.

In the fuel cell stack shown in FIG. 1, the fuel cell stack runs in “closed-end” mode, which means process fluids and coolant are supplied to and discharged from same end of the fuel cell stack. It should be understood that in other versions, the fuel cell may run in “flow-through” mode where process fluids and coolant enter the fuel cell stack from one end and leave the stack from the opposite end. This requires the first end plate 102 be provided with corresponding connection ports for process fluids. It should also be understood that in practice it is useful to stack the several plates 130, 120 and MEAs 124 to form a fuel cell stack to produce a greater current output. Cell stacks may have more than one hundred MEAs 124.

A treatment system is described herein that can be used in a fuel cell test station connected to an electrochemical cell stack. Such stations perform at least two functions. First, the station is used to measure the performance of the cell stack. Second, the station is used to simulate conditions that a cell stack might encounter during a real application.

For example, a fuel cell stack sometimes operates in a cold, dry climate. The humidity of process fluids in such a climate can be significantly lower than the optimal humidity at which the MEA 124 operates. A fuel cell test station can provide low humidity process fluid to a fuel cell stack to test the performance of the stack under less than optimal humidity and temperature levels.

In the treatment system, a source provides process fluids that can have a dew point temperature that is significantly higher than the temperatures encountered upstream in a heating and cooling module of the system. To reduce humidity, the treatment system removes water from the process fluid in the form of ice, and effectively manages the removal thereof to prevent excessive build up in the test station and/or fuel cell stack under test.

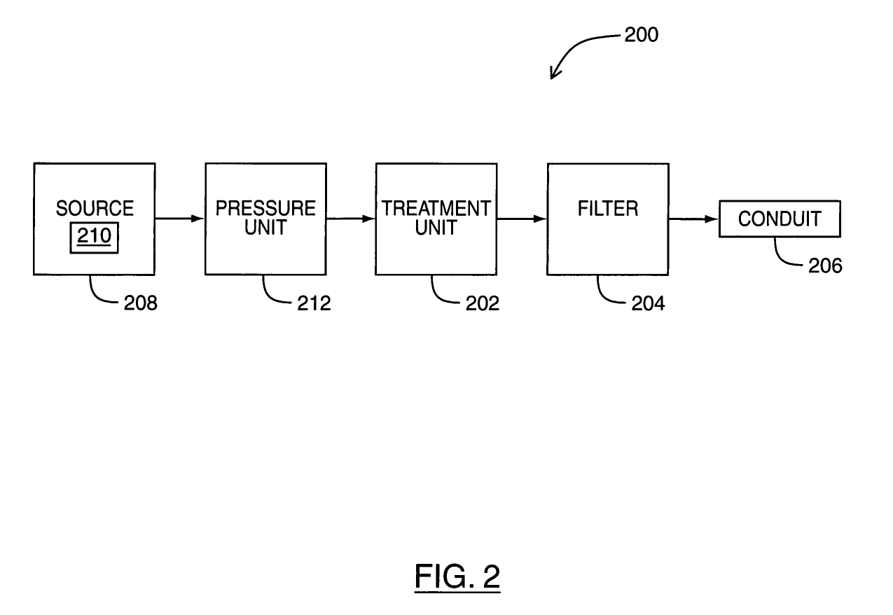

FIG. 2 shows a block diagram of a treatment system 200 for treating process fluids delivered to an electrochemical cell stack with sub-zero temperature and dew point conditions. The system 200 includes a first treatment unit 202, a filter 204, and at least one process fluid conduit 206. The system 200 includes a process fluid source 208 having a humidifier 210. The system 200 further includes a pressure unit 212.

The first treatment unit 202 can treat a process fluid contained therein. For example, the dew point and the temperature of the process fluid can be altered. In particular, the treatment unit 202 can impart a first range of temperatures to the process fluid. The first range includes temperatures below the freezing point of water, which includes the full range of freezing temperatures of water under various pressures likely to be encountered. The first temperature unit 202 can impart a temperature to the process fluid low enough to cause ice particles to form.

The filter 204 removes ice particles larger than a particular size according to the pore size of the filter 204. The process fluid is led through the filter 204 to remove at least a majority of ice particles formed during the temperature and dew point setting of the process fluid in the temperature unit 202. The ice crystals are generally entrained in the process fluid flow, similar to liquid droplets forming an aerosol in air and then freezing. Thus, a large surface area filter 204 with small pores is used for filtering out the ice particles, while keeping the process fluid pressure drop to an acceptable level. The pore size may be approximately 0.1 microns, or whatever size is suitable for the acceptable pressure drop. By removing water in the form of ice, the dew point of the process fluid ultimately delivered to the electrochemical cell stack can be lowered to sub-zero levels.

Separate systems 200 are used for the anode process fluid (fuel) and the cathode process fuel (oxidant). Each system 200 includes at least one process fluid conduit 206 to deliver the filtered process fluid to the cell stack. For the cell stack 100 of FIG. 1, for example, one system 200 includes one process fluid conduit 206 to deliver oxidant to the connection port 106, and another system 200 includes another one process fluid conduit 206 to deliver fuel to the connection port 110. Instead of the open-end mode shown in FIG. 1, the fuel cell may operate in a dead-end mode, in which fuel is supplied to the fuel cell and reacts therein without leaving the fuel cell. The same system 200 can be used in open mode and dead-end mode applications.

The process fluid source 208 supplies process fluids to the treatment unit 202. A humidifier 210 contained therein can humidify the process fluids before delivery to the treatment unit 202.

A pressure unit 212, which can include a back pressure regulation valve, establishes a pressure within a particular pressure range in parts of the process fluid source 208, thus reducing the water content of the process fluids being delivered from the source 208 to the treatment unit 202. Two embodiments are possible in which the position of the pressure unit varies.

In the first embodiment, which is not shown in FIG. 2, the pressure unit 212 is disposed between the filter 204 and the fluid conduit 206 leading to the cell stack 100. The pressure unit 212 is used to keep the pressure high upstream to reduce the water up-take of the process fluid. Thus, when the water-containing process fluid crosses the pressure unit 212, from an area of higher pressure to lower pressure, the water vapour pressure also drops thereby reducing the dew point. However, the higher pressure upstream of the pressure unit 212 has the drawback of decreasing the process fluid response time, and is not necessary in all models of the invention.

In the second embodiment, shown in FIG. 2, the pressure unit 212 is located upstream of the treatment unit 202. This arrangement, which may be used in various models of the invention, has the advantage of lowering the humidity of the fluid delivered to the treatment unit 202. Thus, the water vapour removal demands of the treatment unit 202 are reduced. In addition, with the pressure unit 212 disposed as in FIG. 2, the pressure downstream of the unit 212 is reduced and the fluid velocity increases improving the effectiveness of the treatment unit 202.

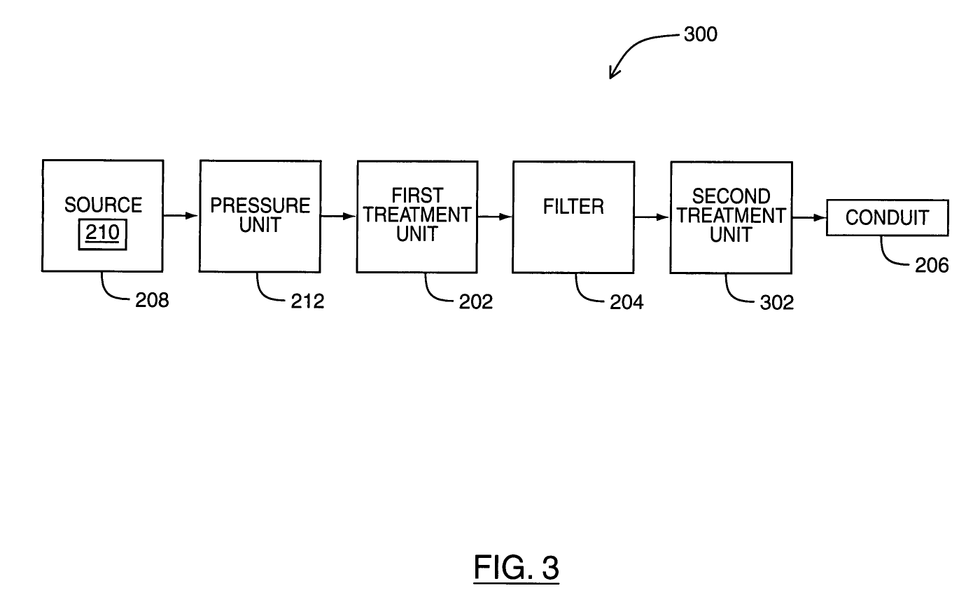

FIG. 3 shows a block diagram of another embodiment of a system 300 for treating process fluids delivered to an electrochemical cell stack. Like the system shown in FIG. 2, the system 300 includes a first treatment unit 202, a filter 204, at least one process fluid conduit 206, a process fluid source 208 with humidifier 210, and a pressure unit 212. In addition, the system 300 includes a second treatment unit 302 disposed between the filter 204 and the conduit 206 leading to the fuel cell stack 100 (not shown in FIG. 3).

The second treatment unit 302 can increase the temperature of the process fluids exiting the filter 204. The temperature increase is typically small, in the range of 5° C. to 30° C., and prevents the formation of any further ice crystals in the process fluid.

Treatment units 202 and 302, and filters 204 become plugged with ice after operating for a matter of hours. When the treatment units 202 and 302, and the filters 204 are warmed above the freezing point of water, the accumulated ice melts to water and drains out without affecting process performance. This method of preventing ice build up is acceptable when stacks are typically only operated in sub-zero conditions for a few hours at a time.

If build-up of ice is a problem, the filter can be heated with an auxiliary heating source to melt accumulated ice. In another embodiment, two or more filters can be used to share the burden of removing ice to limit ice build-up in any one filter.

The catalytic reactions in the MEA 124 require the presence of humidity within the electrodes, but if that humidity freezes, minor damage to the electrode membranes may occur. The filters are rated 95% efficiency for 0.1 micron particles. The pressure of the process fluids between pressure device 212 and treatment device 202 can be monitored and compared with the pressure at the stack inlet, after conduit 206. When the pressure before the treatment unit 202 becomes significantly higher than the pressure after conduit 206, one can infer that the treatment unit 202 or the filter 204 is becoming significantly burdened with ice.

When the fuel cell operating conditions call for dew points above the freezing point of water, the treatment unit 202 and the filter 204 are controlled to the operating gas temperature. This alleviates the potential condensation of water or loss of heat, and thus does not have any appreciable effect on the process fluid conditions.

In typical operation, the humidifier 210 within the fluid source 208 operates in the range of 30° C. dew point at 400 kPa gauge pressure. Thus, the content of water vapour in the gas stream is 0.0759 kg of water per kg of gas. The typical required stack operating conditions have a dew point of −30° C. at 100 kPa gauge. Thus the required content of water vapour in the gas stream is 0.0022 kg of water per kg of gas. In this embodiment, the treatment unit 202 and 204 are required to remove 0.0737 kg of water per kg of gas.

In an embodiment without pressure unit 212 between the process fluid source 208 and the treatment unit 202, the process fluid source 208 produces fluids with higher moisture contents. With humidifier 210 operating in the range of 30° C. dew point at 100 kPa gauge pressure, the content of water vapour in the gas stream is 0.1915 kg of water per kg of gas. Thus to achieve the conditions of −30° C. at 100 kPa gauge in the stack, the treatment unit 202 and 204 is required to remove 0.1893 kg of water per kg of gas. Since the removal rate of water is twice as high, a test station would be able to operate for half the time before the treatment unit 202 and filter 204 became plugged with ice.

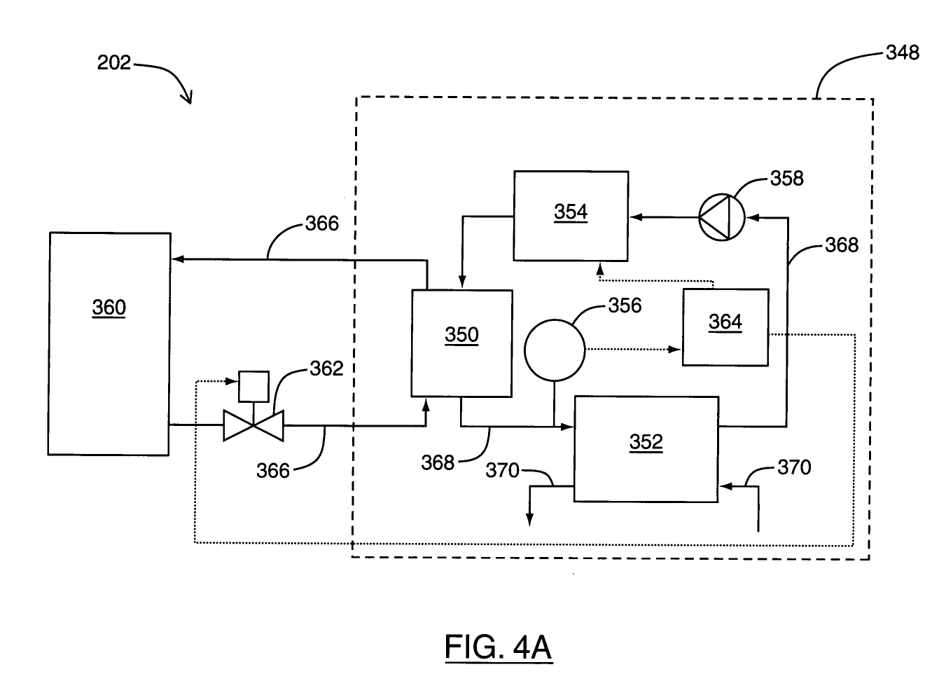

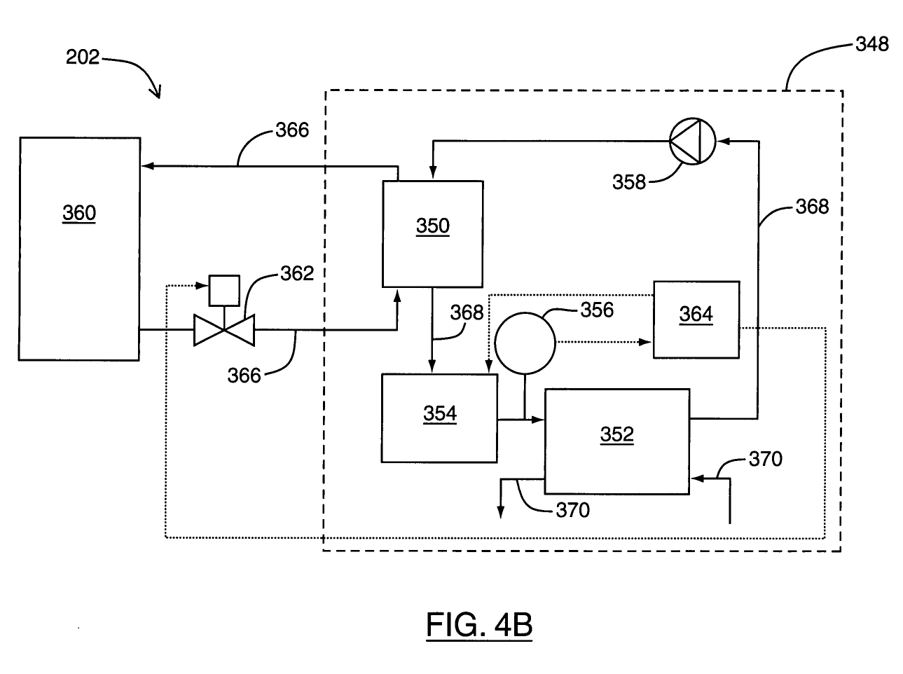

FIG. 4A shows the treatment unit 202 of FIG. 2. The treatment unit 302 of FIG. 3 is similar. The treatment unit 202 includes a heating and cooling circulation module 348, a chiller device 360 and a flow control valve 362. The heating and cooling circulation module 348 includes a first heat exchanger 350, a second heat exchanger 352, a heater 354, a temperature sensor 356, a pump 358 and a temperature control unit 364.

The chiller device 360 supplies the first heat exchanger 350 with a first heat exchange fluid (not shown), such as oil, to draw heat therefrom. In particular, the first heat exchange fluid circulates around a first loop 366 transferring heat from the first exchanger 350 to the chiller device 360.

A second loop 368 depicts the motion of a second heat exchange fluid (not shown), which can also be oil, for example. The temperature of the second heat exchange fluid flowing through the second loop 368 is regulated by adjusting the flow rate of the first heat exchange fluid from the chiller device 360 through the first heat exchanger 350 using the flow control valve 362. The flow control valve 362 and the temperature sensor 356 enable a control device 364 to regulate heat exchange.

The pump 358 can be of the centrifugal type or of the type providing positive displacement. The pump 358 has sufficient pumping capacity for the large variations in viscosity that a typical heat exchange fluid can display within the intended operating temperature range (down to −40° C., and up to 120° C.).

A temperature control unit 364 regulates the temperature of the second heat exchange fluid either heated in the heater 354 or cooled in the first heat exchanger 350, and then used in the second heat exchanger 352. A third loop 370, only part of which is shown, depicts the flow of process fluid (not shown) from the process fluid source 208 (shown in FIGS. 2 and 3, but not FIG. 4A or 4B) to the conduit 206 (shown in FIGS. 2 and 3, but not FIG. 4A or 4B). The process fluid exchanges heat with the second heat exchange fluid of the second heat exchanger 352.

The first heat exchange fluid is optimized for efficient heat transfer to the heating or cooling devices (not shown) of the chiller device 360, while the second heat exchange fluid is optimized for efficient heat transfer from the process fluid. The flow through the heat exchanger 352 is set up for counter-flow. Thus, the temperature of the fluid exiting the heat exchanger 352 is close to the temperature of the incoming fluid as measured by the sensor 356. The heat capacity of the transfer fluid in loop 368 is significantly higher than the heat capacity of the process fluid and the heat exchanger is designed to maintain turbulent flow, so the exiting temperature of the process fluid will match the transfer fluid temperature.

A variant of the treatment unit embodiment of FIG. 4A is shown as a block diagram in FIG. 4B. The components are the same, but the relative arrangement is different. In particular, in the embodiment of FIG. 4B, the heater 354 is disposed upstream of the first heat exchanger 350. The choice between the embodiment shown in FIG. 4A and that shown in FIG. 4B can be influenced by the layout that best suits the space available.

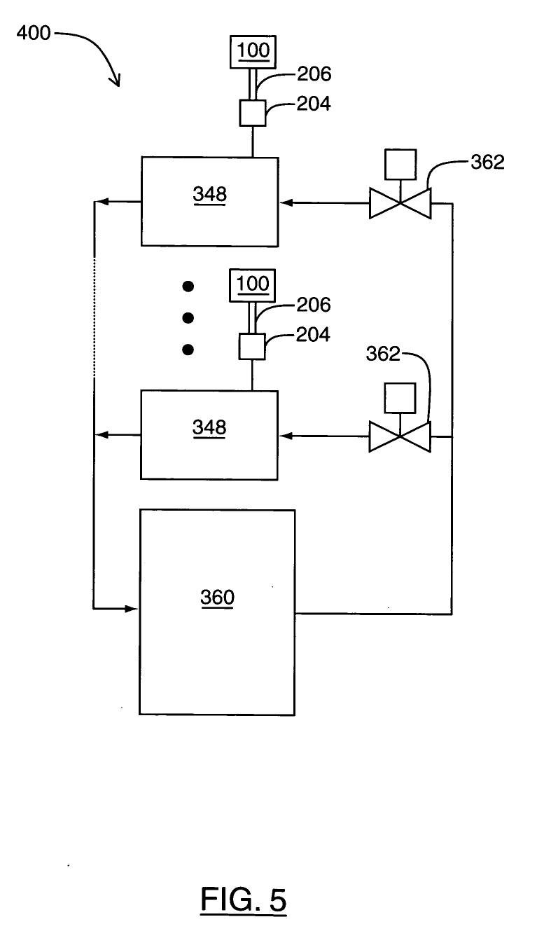

In a particularly convenient embodiment, one chiller device can service several heating and cooling modules. A block diagram of a system 400 corresponding to such an embodiment is shown in FIG. 5. The system 400 includes a chiller device 360 connected to a plurality of heating and cooling modules 348 via corresponding flow control valves 362. Each heating and cooling circulation module 348 is coupled to a cell stack 100 via a process fluid conduit 206. Filters 204 are disposed downstream of the heating and cooling modules 348.

Each one of the heating and cooling modules 348 is capable of independently setting a temperature of a process fluid contained therein. To each heating and cooling circulation module 348 is associated a filter 204. Each filter 204 removes ice particles formed when the associated heating and cooling circulation module 348 imparts a temperature to the process fluid low enough to cause ice particles to form. The process fluid conduits 206 deliver filtered process fluid from the filters 204 to an associated electrochemical cell stack 100, which cell stack 100 is shown in more detail in FIG. 1. The chiller device 360 removes heat from the heating and cooling modules 348.

In particular, the chiller device 360 supplies the remote heating and cooling modules 348 with a heat exchange fluid, such as oil, to remove heat therefrom. Pressure units (not shown in FIG. 5) can be employed as described above. The description of the heating and cooling modules 348 appears above with reference to FIGS. 4A and 4B, and is not repeated.

The use of a central chiller device 360 servicing several heating and cooling modules 348 has several advantages. First, there is a cost savings because only one chiller device 360 is used instead of one for each cell stack. Second, because with proper insulation the chiller device 360 can be remote from the heating and cooling module 348—the device 360 and module 348 can be as far as 5 m or more—the chiller device can be conveniently serviced at one location, instead of having to access the device at the many locations of the heating and cooling modules 348. Also, the management of the chiller device 360 can more easily be divorced from that of the heating and cooling modules 348. Thus, the management and maintenance of the chiller device 360 can be left to a third party provider. Such an arrangement can reduce costs because it obviates the need for operators of electrochemical cell stacks or test stations operated in groups to purchase and service individual chiller devices. Instead, chiller device service can possibly be outsourced to the third party provider.

It is anticipated that those having ordinary skills in the art can make various modifications to the embodiments disclosed herein after learning the teaching of the present invention. For example, although emphasis has been placed above on treatment systems for use in the context of testing cell stacks in sub-zero conditions, more generally, the treatment systems can treat process fluids to better optimize the properties of process fluids entering the cell during normal operation. Thus, the treatment systems described above can be used to alter the properties of process fluids, such as by raising or lowering at least one of temperature, pressure and humidity to improve the performance of the cell stack.

It is also to be appreciated that the present invention is primarily concerned with the treatment of process gases in a test station for fuel cell stacks or power modules. When testing cold start capability of such fuel cell stacks, it is necessary to supply process gases at sub-zero temperatures, so as to test the behaviour of the fuel cell stack under conditions in which ice could form within the stack, blocking flow passages etc. However, it is also recognized that once started and operating, a fuel cell stack would usually be allowed to warm up to a temperature well above 0° C., so that the problem of potential ice build up is avoided. As such, test stations will often only need to be capable of supplying process gases at below zero temperatures for relatively short time periods, during which build of ice particles in a filter can be tolerated.

It is also to be understood that the invention is also expected to have applicability as part of the “balance of plant” for a fuel cell power system, so as to provide a cold start capability. As such, the invention has applicability to a wide range of fuel cells including, in addition to PEM (Proton Exchange membrane) fuel cells, any other fuel cell requiring a gas or process fluid to be conditioned in such a way that moisture may condense out and form ice particles.

A fuel cell test station is a device that provides the necessary balance of plant to run a fuel cell stack or power module, and usually includes: means for supplying fuel and oxidant gases at desired flow rates and pressures; means for conditioning the process gases to give them desired temperatures and humidity levels; vents or exhausts for process gases and/or recycling of these gases as required; a load for absorbing power generated by the fuel cell stack; supply connections for supply of a coolant at a desired flow rate and temperature; various monitoring devices and sensors including, for example, measurement of overall current and voltage generated by the fuel cell stack, monitoring of voltage across individual cells, monitoring process gas conditions at inlet and/or outlet of the cell stack, monitoring coolant flow rates and temperatures; and an enclosure for containing a fuel cell stack or power module. A power module usually is a fuel cell stack with associated balance of plant components need for operation, so that the functions required from the test station are reduced.

In addition, the number and arrangement of components in the system might be different, and different elements might be used to achieve the same specific function. However, these modifications should be considered to fall under the scope of the invention as defined in the following claims.

1. A system for treating a process fluid delivered to an electrochemical cell stack, the system comprising
a first treatment unit capable of treating and imparting a first range of temperatures to a process fluid, said first range including a temperature below the freezing point of water;
a filter for removing ice particles larger than a particular size formed when the first treatment unit imparts a temperature to the process fluid low enough to cause said ice particles to form; and
at least one process fluid conduit for delivering the filtered process fluid to the cell stack.

2. The system of claim 1, further comprising an electrochemical cell stack coupled to the first treatment unit via the at least one process fluid conduit.

3. The system of claim 1, further comprising a process fluid source for supplying the process fluid to the first treatment unit.

4. The system of claim 1, further comprising a second treatment unit downstream of the filter capable of imparting a second range of temperatures to the process fluid.

5. The system of claim 4, wherein the second treatment unit raises the temperature of the process fluid.

6. The system of claim 5, wherein the second treatment unit raises the temperature of the process fluid by about five to thirty degrees Celsius.

7. The system of claim 1, wherein the first treatment unit includes
a heating and cooling module for heating and cooling the process fluid;
a chiller device through which a first heat exchange fluid passes for removing heat from the heating and cooling module; and
a flow control valve between the heating and cooling module and the chiller device for regulating the flow of the first heat exchange fluid therebetween.

8. The system of claim 7, wherein the heating and cooling module includes
a first heat exchanger for transferring heat to the first heat exchange fluid;
a second heat exchanger coupled to the process fluid for exchanging heat therebetween;
a heater for heating a second heat exchange fluid flowing through the first heat exchanger and the second heat exchanger;
a temperature sensor for sensing the temperature of the second heat exchange fluid;
a pump for pumping the second heat exchange fluid; and
a temperature control unit for controlling the temperature of the second heat exchange fluid.

9. The system of claim 1, further comprising a pressure unit for establishing a pressure within a particular pressure range.

10. The system of claim 9, wherein the pressure unit includes a back pressure regulation valve.

11. The system of claim 9, wherein the pressure unit is downstream of the filter.

12. The system of claim 9, wherein the pressure unit is upstream of the first treatment unit.

13. A system for treating process fluids for delivery to a plurality of electrochemical cell stacks, the system comprising
a plurality of heating and cooling modules, each one capable of independently setting a temperature of a process fluid therein;
a plurality of filters, each one associated with one respective heating and cooling module and each one functioning to remove ice particles formed when the associated heating and cooling module imparts a temperature to the process fluid low enough to cause said ice particles to form;
a plurality of process fluid conduits, each one, in use, transporting filtered process fluid from one of the filters to an associated electrochemical cell stack; and
only one chiller device for removing heat from the plurality of heating and cooling modules.

14. The system of claim 13, in combination with a plurality of fuel cell test stations, wherein each of the fuel cell test stations includes one of the heating and cooling modules, one of the filters and one of the process fluid conduits and wherein the common chiller device is connected the plurality of fuel cell test stations and the heating and cooling modules included therein.

15. The system of claim 14, wherein the chiller device includes a heat exchange fluid that circulates through the chiller device and each heating and cooling module.

16. The system of claim 15, further comprising a plurality of flow control valves for regulating the circulation of heat exchange fluid through the chiller device and the heating and cooling modules.

17. The system of claim 15, wherein each heating and cooling module includes
a first heat exchanger for transferring heat to the first heat exchange fluid transported to the chiller device;
a second heat exchanger coupled to the process fluid for exchanging heat therebetween;
a heater for heating a second heat exchange fluid circulating through the first heat exchanger and the second heat exchanger;
a temperature sensor for sensing the temperature of the second heat exchange fluid;
a pump for pumping the second heat exchange fluid; and
a temperature control unit for controlling the temperature of the second heat exchange fluid.

18. The system of claim 14, further comprising a plurality of pressure units, each one disposed between the respective filter and, in use, the electrochemical cell stack, for establishing pressure of the process fluids.

19. The system of claim 18, wherein each pressure unit includes a back pressure regulation valve.

20. The system of claim 19, wherein each pressure unit is upstream of the respective heating and cooling module.

21. A test station for testing an electrochemical cell stack, the test station comprising
a first treatment unit capable of treating and imparting a first range of temperatures to a process fluid, said first range including a temperature below the freezing point of water;
a filter for removing ice particles larger than a particular size formed when the first treatment unit imparts a temperature to the process fluid low enough to cause said ice particles to form; and
at least one process fluid conduit for delivering the filtered process fluid to the cell stack.

22. The test station of claim 21, further comprising a process fluid source for supplying the process fluid to the first treatment unit.

23. The test station of claim 21, further comprising a second treatment unit downstream of the filter capable of imparting a second range of temperatures to the process fluid.

24. The test station of claim 23, wherein the second treatment unit raises the temperature of the process fluid.

25. The test station of claim 24, wherein the second treatment unit raises the temperature of the process fluid by about five to thirty degrees Celsius.

26. The test station of claim 21, wherein the first treatment unit includes
a heating and cooling module for heating and cooling the process fluid;
a chiller device through which a first heat exchange fluid passes for removing heat from the heating and cooling module; and
a flow control valve between the heating and cooling module and the chiller device for regulating the flow of the first heat exchange fluid therebetween.

27. A method of treating a process fluid, to be delivered to an electrochemical cell stack, the method comprising:
cooling the fluid to a temperature below the freezing point of water;
filtering out any ice particles that have formed due to the cooling step; and
delivering the process fluid to the electrochemical cell stack.
